(19) United States
(12) Patent Application Publication
(10) Pub. No.: US 2005/0042223 A1
(43) Pub. Date: Feb. 24, 2005

(54) Hybridomas producing high levels human sequence antibody

(75) Inventors: S. Edward Lee, Waterford, CT (US); William R. Hermans, Millbury, MA (US); David J. Wasilko, Oakdale, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/673,738

(22) Filed: Sep. 29, 2003

Related U.S. Application Data
(60) Provisional application No. 60/415,088, filed on Sep. 30, 2002.

(51) Int. Cl. A61K 39/395; C12N 5/06; C07K 16/30

(52) U.S. Cl. 424/155.1; 435/344; 530/388.8

(57) ABSTRACT

A hybridoma cell line producing high levels of human sequence antibody. Also described is a hybridoma cell line producing human sequence antibodies that is adapted for growth in serum-free media or animal-derived-components-free media. A hybridoma cell line producing anti-CTLA4 antibodies is most preferred.

This application claims the benefit of U.S. Provisional Application 60/415,088 filed Sep. 30, 2002, which application is hereby incorporated by reference, herein.

### BACKGROUND OF THE INVENTION

The invention relates to the development of hybridoma cell lines producing high levels of human sequence monoclonal antibodies where the cell lines are adapted to grow in media lacking serum and other animal-derived components. Hybridomas of the present invention may be employed in the large-scale production of such antibodies.

As many as one-third of all biotechnology products currently in development are MAbs. Understandably, there is an increasing demand for large quantities of these MAbs. Unfortunately, animal cell culture often does not readily meet this increased demand. The cellular machinery of an animal cell (versus a bacterial cell) generally is required in order for complex macromolecules such as MAbs to be produced. However, as compared with bacterial cultures, animal cell cultures have low production rates and typically generate low production yields (Bierau et al., J Biotechnology 62: 195-207, 1998). As a result, the need to produce large quantities of MAbs from animal cell cultures often goes unmet.

Furthermore, animal cell cultures typically require complex media containing animal serum, and other undefined growth factors or animal-derived components. This is particularly true for hybridoma cell cultures in which a medium usually contains fetal bovine serum (FBS). Even if an original culture can be optimized for the production of a desired MAb, the presence of animal-derived components in the culture media translates into increased costs for producing the MAb. In addition, regulatory approval for the use of the desired MAb may be withheld if the macromolecule is produced in cultures containing animal-derived components. Regulatory authorities must consider the possibility that disease-causing pathogens may be transmitted from the animal-derived components. The use of media lacking components of animal origin is thus desirable from the perspectives of both cost and safety.

CTLA4 is involved in T-cell activation, which requires two signals: one is antigen-specific and based on T-cell receptor recognition of a major histocompatibility complex (MHC)-peptide coupling, and the second is antigen-nonspecific and delivered by specific T-cell receptors after ligation with their ligands (costimulatory molecules) expressed by antigen-presenting cells (APCs). Engagement of the B7 family of molecules on APCs with their T-cell associated ligands, CD28 and CTLA4 (also called CD152), provides pivotal costimulatory signals in T-cell activation.

Given the roles for CTLA4 in modulating T cell activation, uses for MAbs that have an affinity for CTLA4 are numerous. Not only can these MAbs be used for the development of diagnostic kits and procedures, they are also useful in immunotherapeutic treatments that target binding between CTLA4 and B7 isoforms.

Published international patent applications WO 00/37504 and WO 01/14424 refer to anti-CTLA4 MAbs. U.S. application Ser. No. 10/153,382, filed May 22, 2002 refers to methods of treatment of cancer using CTLA4 antibodies.

### BRIEF SUMMARY OF THE INVENTION

The present invention relates to a hybridoma, wherein said hybridoma produces at least 200 mg/L of a human sequence antibody when cultured in batch culture. In a preferred embodiment, the hybridoma produces more than 300 mg/L, and more preferably more than 390 mg/L of the antibody. The present invention also relates to hybridomas producing high levels of human sequence MAb in media free of animal-derived components.

In one embodiment, the present invention provides hybridomas producing at least 200 mg/L of anti-CTLA4 human sequence antibody when cultured in batch culture. In another embodiment, the invention provides a hybridoma producing at least 300 mg/L, or in other embodiments, at least 390 mg/L, of anti-CTLA4 antibody when cultured in batch culture, particularly wherein hybridoma cultures are maintained in serum-free media or media free of animal-derived components.

The present invention also provides a hybridoma having identifying characteristics of a hybridoma deposited at ATCC deposit number PTA-4537, such as one derived from that hybridoma.

In another aspect, the invention relates to a cell culture comprising a hybridoma producing an antibody having a human amino acid sequence in medium that is substantially serum free, or that is substantially free of animal derived components. The invention also relates to a method of producing antibody from the culture by growing the culture under conditions suitable for expression of the antibody, and isolating the antibody from the culture. In a preferred embodiment, the antibody is a CTLA4 antibody.

In another aspect, the invention provides a culture medium (herein designated as eRDF-ACF Medium) free of animal-derived components, the medium comprising 2 to 10 g/L (or preferably 4 to 9, or most preferably 6.6 g/L) Wheat Gluten Peptides, 0.85 to 0.99× (or preferably 0.9 to 0.99, or most preferably 0.95×) eRDF Stock without HEPES Buffer, 0 to 0.5% (or preferably 0.05 to 0.3, or most preferably 0.095%) Glycerol, 0 to 2× (or preferably 0.5 to 1.5, or most preferably 0.95×) 2-mercaptoethanol Stock, 0.5 to 8 mM (or preferably 1 to 4, or most preferably 1.9 mM) L-Glutamine, 0.5 to 5× (or preferably 0.8 to 3, or most preferably 0.95×) IES Stock, 2 to 8 μM (or preferably 4 to 6, or most preferably 4.7 μM) Ascorbic Acid, 1 to 5 mM (or preferably 1.5 to 4, or most preferably 1.9 mM) Ferric Citrate, 2 to 10×10⁻⁴× (or preferably 4 to 6, or most preferably 4.7×10⁻⁴×) Lipid Stock, 0 to 0.5% (or preferably 0.05 to 0.2, or most preferably 0.095%) Medical Antifoam C Emulsion, and 0 to 4× (or preferably 0.5 to 2, or most preferably 0.95×) Pluronic F-68 (10% Stock=100×).

In another aspect, the invention provides an anti-CTLA4 antibody delivery system comprising a cell culture medium and a hybridoma selected from the group consisting of hybridoma deposited at American Type Culture Collection (ATCC) deposit number PTA-4537 and hybridoma producing at least 200 mg/L, or in other embodiments, at least 300 mg/L, or in other embodiments, at least 390 mg/L of anti-CTLA4 antibody when cultured in batch culture, particularly wherein hybridoma cultures are maintained in serum-free cell culture media or cell culture media free of animal-derived components. Alternately, the hybridoma employed in the delivery system can be derived from these hybridomas.

In particular preferred embodiments, the invention provides such anti-CTLA4 antibody delivery systems wherein the cell culture medium is an eRDF-ACF medium free of animal-derived components, the medium comprising 2 to 10 g/L Wheat Gluten Peptides, 0.85 to 0.99× eRDF Stock without HEPES Buffer, 0 to 0.5% Glycerol, 0 to 2×2-mercaptoethanol Stock, 0.5 to 8 mM L-Glutamine, 0.5 to 5× IES Stock, 2 to 8 μM Ascorbic Acid, 1 to 5 mM Ferric Citrate, 2 to 10×10⁻⁴× Lipid Stock, 0 to 0.5% Medical Antifoam C Emulsion, and 0 to 4× Pluronic F-68 (10% Stock=100×).

In another aspect, the invention provides a method of making anti-CTLA4 antibody, the method comprising: i) admixing a cell culture medium and a hybridoma selected from the group consisting of a hybridoma deposited at ATCC deposit number PTA-4537 and hybridoma producing at least 200 mg/L, or in other embodiments, at least 300 mg/L, or in other embodiments, at least 390 mg/L of anti-CTLA4 antibody when cultured in batch culture, particularly wherein hybridoma cultures are maintained in serum-free cell culture media or cell culture media free of animal-derived components, to form an admixture; ii) culturing the admixture; and iii) obtaining anti-CTLA4 monoclonal antibody from the cultured admixture. Alternately, the hybridoma can be derived from the hybridomas listed in step (i).

In another aspect, the invention relates to a pharmaceutical composition for the treatment of cancer in a mammal comprising an amount of an anti-CTLA4 antibody produced by a hybridoma of the invention that is effective in treating said cancer and a pharmaceutically acceptable carrier. The invention also relates to a method for treating cancer in a mammal comprising administering to said mammal an amount of an antibody produced by a hybridoma of the invention that is effective in treating said cancer.

### DETAILED DESCRIPTION OF THE INVENTION

All publications and patent references listed herein are hereby incorporated by reference in their entireties.

Definitions and Abbreviations

APC—antigen presenting cell

ATCC—American Type Culture Collection

“Batch culture” of hybridomas is used herein to refer to a culture process in which cells are admixed with a liquid medium and are allowed to grow, metabolize, and replicate in that medium without medium being replaced, added or removed in the culture process (other than by passive condensation or evaporation); “batch culture” of hybridomas does not include culture in hollow-fiber perfusion systems, fluidized bed reactors, or similar culture processes or systems in which medium is refreshed during culture (for descriptions of hollow-fiber perfusion systems, fluidized bed reactors, or similar culture processes or systems; see Freshney “Culture of animal cells: a manual of basic technique,” 4th ed., Wiley-Liss, New York, N.Y., 2000).

BBS—borate buffered saline

BSA—bovine serum albumin

CS—hybridoma fusion and cloning supplement

DMEM—Dulbecco's Modified Eagle Medium

DMSO—dimethyl sulfoxide

ELISA—enzyme-linked immunosorbent assay

FBS—fetal bovine serum

IL6—interleukin-6

MAb—monoclonal antibody

MHC—major histocompatibility complex

PBS—phosphate buffered saline

“Wheat Gluten Peptides” is used herein to refer to preparations of peptide compositions from wheat gluten, such as by enzymatic digestion; one example is a preparation represented by HYPEP® 4602 (QUEST INTERNATIONAL, 5115 Sodge Blvd., Hoffman Estates, Ill., 60192, United States; Cat. No. 5Z10471), which is an enzymatic digest of wheat gluten that provides a high quality source of peptides, and is particularly rich in stable glutamine-containing peptides; it contains relatively high levels of free amino acids (e.g., 28%), as well as di- and tri-peptides; and it was designed for tissue culture applications as an amino acid and glutamine source and/or as a bovine serum replacer.

In one aspect, a particular advantage of hybridomas of the present invention is their capacity to thrive and produce 200 mg/L or more of human sequence antibody in batch culture, wherein hybridoma cultures are maintained in serum-free media or media free of animal-derived components. The capacity of hybridomas of the present invention to thrive in media free of animal-derived components is particularly desirable for reducing costs associated with hybridoma culture and for obtaining purified MAb preparations that are assuredly pathogen-free.

The hybridoma of the present invention expresses human immunoglobulin genes. Use of transgenic mice is known in the art to product such “human sequence” antibodies. One such method is described in Mendez et al. Nature Genetics 15:146-156 (1997), Green and Jakobovits J. Exp. Med. 188:483-495 (1998), and U.S. Patent Application Ser. No. 08/759,620 (filed Dec. 3, 1996). The use of such mice to obtain human antibodies is also described in U.S. patent applications Ser. No. 07/466,008 (filed Jan. 12, 1990), U.S. Ser. No. 07/610,515 (filed Nov. 8, 1990), U.S. Ser. No. 07/919,297 (filed Jul. 24, 1992), U.S. Ser. No. 07/922,649 (filed Jul. 30, 1992), filed U.S. Ser. No. 08/031,801 (filed Mar. 15,1993), U.S. Ser. No. 08/112,848 (filed Aug. 27, 1993), U.S. Ser. No. 08/234,145 (filed Apr. 28, 1994), U.S. Ser. No. 08/376,279 (filed Jan. 20, 1995), U.S. Ser. No. 08/430, 938 (filed Apr. 27, 1995), U.S. Ser. No. 08/464,584 (filed Jun. 5, 1995), U.S. Ser. No. 08/464,582 (filed Jun. 5, 1995), U.S. Ser. No. 08/463,191 (filed Jun. 5, 1995), U.S. Ser. No. 08/462,837 (filed Jun. 5, 1995), U.S. Ser. No. 08/486,853 (filed Jun. 5, 1995), U.S. Ser. No. 08/486,857 (filed Jun. 5, 1995), U.S. Ser. No. 08/486,859 (filed Jun. 5, 1995), U.S. Ser. No. 08/462,513 (filed Jun. 5, 1995), U.S. Ser. No. 08/724,752 (filed Oct. 2, 1996), and U.S. Ser. No. 08/759,620 (filed Dec. 3, 1996). See also Mendez et al. Nature Genetics 15:146-156 (1997) and Green and Jakobovits J. Exp. Med. 188:483-495 (1998). See also European Patent EP 0 463 151 (grant published Jun. 12, 1996), International Patent Application WO 94/02602 (published Feb. 3, 1994), International Patent Application WO 96/34096 (published Oct. 31, 1996), and WO 98/24893 (published Jun. 11, 1998). Also see international patent publications WO 00/37504 and WO 01/14424 describing such human sequence anti-CTLA4 antibodies produced using such mice.

Generation of hybridomas producing human sequence antibodies is accomplished according to the above references. As shown further in the examples below, such hybridomas are adapted for production and serum-free growth according to the methods described.

In a further embodiment of the invention, the culture containing the hybridoma produces more than 300 and less than 600 mg/L of antibody.

The CTLA4 antibody produced according to one embodiment of the invention is useful for example, for the treatment of cancer. Examples of such cancers include lung cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, colon cancer, breast cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, renal cell carcinoma, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, t-cell lymphoma, environmentally induced cancers including those induced by asbestos, and combinations of those cancers.

The antibodies can be incorporated into pharmaceutical compositions suitable for administration to a subject. Typically, the pharmaceutical composition comprises the antibody and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, and the like, as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the antibody.

The antibodies may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies can be administered by a variety of methods known in the art, including, without limitation, oral, parenteral, mucosal, by-inhalation, topical, buccal, nasal, and rectal. For many therapeutic applications, the preferred route/mode of administration is subcutaneous, intramuscular, intravenous or infusion. Non-needle injection may be employed, if desired. As will be appreciated by the skilled artisan, the route and/or mode of administration will vary depending upon the desired results.

In certain embodiments, the antibody may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, the antibody may be orally administered, for example, with an inert diluent or an assimilable edible carrier. The antibody (and other ingredients, if desired) may also be enclosed in a hard or soft shell gelatin capsule, compressed into tablets, or incorporated directly into a patient's diet. For oral therapeutic administration, the antibodies may be incorporated with excipients and used in the form of ingestible tablets, buccal tablets, troches, capsules, elixirs, suspensions, syrups, wafers, and the like. To administer the antibody by other than parenteral administration, it may be necessary to coat it with, or co-administer the compound with, a material to prevent its inactivation.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the antibody and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of an antibody administered according to the invention is 0.1-100 mg/kg, more preferably 0.5-50 mg/kg, more preferably 1-20 mg/kg, and even more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In one embodiment, the antibody is administered in an intravenous formulation as a sterile aqueous solution containing 5 or 10 mg/ml of antibody, with 20 mM sodium acetate, 0.2 mg/ml polysorbate 80, and 140 mM sodium chloride at pH 5.5.

In one embodiment, part of the dose is administered by an intraveneous bolus and the rest by infusion of the antibody formulation. In yet another embodiment, a 0.01 mg/kg intravenous bolus injection of the antibody is followed by a 0.1 mg/kg intravenous injection over 3-5 minutes, followed by a 1 and 3 mg/kg infusion in 100 ml saline at 100 ml/hour, followed by a 4 to 10 mg/kg infusion in 250 ml saline at 100 ml/hour, followed by a 12.5 to 21 mg/kg infusion in 500 ml saline at 100 ml/hour, followed by a 28 mg/kg infusion in 600 ml saline (500+100 bags) at 120 ml/hour.

When administered to treat cancer, the antibody can be combined with an agent selected from the group consisting of a chemotherapeutic agent, a cancer vaccine, an immunomodulatory agent, an anti-angiogenesis agent, an anti-vascular agent, a signal transduction inhibitor, an antiproliferative agent, an apoptosis inducer, and an inhibitor of a survival pathway. Further description of administration of such antibodies is described in International patent applications WO 00/37504, WO 01/14424, and U.S. application Ser. No. 10/153,382, filed May 22, 2002, the texts of which are incorporated by reference herein.

The antibody can be labeled. This can be done by incorporation of a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

The invention is illustrated by the following examples, which are not intended to be construed to limit the claims.

### EXAMPLE 1

### eRDF-ACF Medium

eRDF-ACF Medium is a serum-free medium; it contains no animal-derived components. eRDF-ACF Medium was made by combining Wheat Gluten Peptides (e.g., HYPEP®4602; 2-10 g/L, preferred 4-9 or 6.6 g/L), eRDF Stock w/o HEPES Buffer (0.85-0.99×, preferred 0.9-0.99 or 0.95×), Glycerol (0-0.5%, preferred 0.05-0.3 or 0.095%), 2-Mercaptoethanol Stock (0-2×, preferred 0.5-1.5 or 0.95×), L-Glutamine (0.5-8 mM, preferred 1-4 or 1.9 mM), IES Stock (0.5-5.0×, preferred 0.8-3 or 0.95×), Ascorbic Acid (2-8 μM, preferred 4-6 or 4.7 μM), Ferric Citrate (1-5 mM, preferred 1.5-4 or 1.9 mM), Lipid Stock (2-10×10⁻⁴×, preferred 4-6'10⁻⁴ or 4.7×10⁻⁴×), Medical Antifoam C Emulsion (0-0.5% preferred 0.05-0.2 or 0.095%), and Pluronic F-68 (i.e., 10% Stock=100×; 0-4×, preferred 0.5-2 or 0.95×).

A particularly preferred composition is show below. Suppliers, catalog numbers, and additional information for eRDF-ACF Medium are as follows:

Wheat Gluten Peptides (HYPEP® 4602, QUEST INTERNATIONAL, 5115 Sodge Blvd., Hoffman Estates, Ill., 60192, United States; Cat. No. IPL #5Z10471). The amino acid distribution (mg/lg) for the HYPEP® 4602 used, which is a representative distribution (mg/g), is as follows:

| Amino acid | Total | Free |
|---|---|---|
| Alanine | 23 | 9.9 |
| Arginine | 30 | 14 |
| Aspartic Acid | 5 | 2.5 |
| Asparagine | 19 | 4.2 |
| Cysteine | 11 | 0 |
| Glutamic Acid | 71 | 9.6 |
| Glutamine | 284 | 70 |
| Glycine | 28 | 4.6 |
| Histidine | 17 | 7.8 |
| Isoleucine | 35 | 19 |
| Leucine | 67 | 42 |
| Lysine | 11 | 6.2 |
| Methionine | 11 | 7.8 |
| Phenylalanine | 47 | 17 |
| Proline | 116 | 0.7 |
| Serine | 50 | 19 |
| Threonine | 23 | 9.7 |
| Tyrosine | 30 | 13 |
| Valine | 38 | 23 |

Other typical data for chemical characteristics (%) of the HYPEP® 4602 used are as follows: Amino Nitrogen (AN): 4.0; Total Nitrogen (TN): 13.3; AN/TN: 30; Moisture: less than 9.0; Free Amino Acids: 28; Sodium: 0.5; Potassium: 0.1; Calcium: 0.1. Other data for chemical characteristics of the HYPEP® 4602 used are: pH (10% solution): 5.4; and solubility (at 25° C.): 300 g/L. Microbiological characteristics of the HYPEP® 4602 used are: Total viable count: max. 1,000/g; Yeast and Molds: max. 100/g; Salmonella: absent in 25 g; Escherichia coli: absent in 1/g; Staph. aureus: max 10/g.

eRDF Stock w/o HEPES Buffer (LIFE TECHNOLOGIES Cat. No. 000-0348).

Glycerol (SIGMA-ALDRICH Cat. No. 2025).

2-mercaptoethanol Stock (1000×) (LIFE TECHNOLOGIES Cat No. 21985-023).

L-Glutamine Stock (200 mM): 29.2 mg L-glutamine/mL in 0.85% NaCl (LIFE TECHNOLOGIES, Cat. No. 25030-081).

IES Stock (100×):

| Component | Amount | |
|---|---|---|
| Recombinant Insulin (SIGMA-ALDRICH Cat. No. | 100 | mg |
| I-2767) | | |
| Ethanolamine (SIGMA-ALDRICH Cat. No. E-0135) | 19.6 | μL |
| Sodium Selenite Stock Solution (of 1 mg Sodium | 1 | mL |
| Selenite (SIGMA-ALDRICH Cat. No. S-9133) in | | |
| 14.9 mL EBSS) | | |
| Sodium Pyruvate (LIFE TECHNOLOGIES Cat. No. | 1.1 | g |
| 11840-048) | | |
| EBSS (i.e., Earl's Buffered Salts Solution) Stock* | q.s. 100 | mL |

*Earl's Buffered Salts Solution (EBSS) Stock:

| Component | Amount |
|---|---|
| KCl | 40 mg |
| NaCl | 680 mg |
| NaHCO3 | 220 mg |
| NaH2PO4.H2O | 14 mg |
| Glucose | 100 mg |

EBSS Stock was prepared by adding to the components picopure distilled H₂O to a volume near 100 mL and adjusting the pH to 7.2-7.4 (preferably 7.25) with 10% HCl before bringing the final volume to 100 mL with picopure distilled H₂O.

5 mM Ascorbic Acid Stock

| Component | Amount |
|---|---|
| Ascorbic Acid (SIGMA-ALDRICH Cat. No. A-2174) | 88.7 mg |

5 mM Ascorbic Acid Stock was prepared by adding to the 88.7 mg ascorbic acid picopure distilled H₂O to 100 mL and filtered through a 0.2 micron membrane filter (CORNING Cat. No. 430757).

Ferric Citrate Stock (200 mM)

| Component | Amount |
|---|---|
| Ferric Citrate (SIGMA-ALDRICH Cat. No. F-3388) | 489.4 mg |

Ferric Citrate Stock was prepared by adding to the 489.4 mg ferric citrate picopure distilled H₂O to 100 mL, mixing, incubating the mixture in a 37° C. water bath for 6-7 hours with occasional vortexing, and filtering the clear mixture through a 0.2 μm membrane filtration unit (CORNING Cat. No. 430757). The Ferric Citrate Stock was stored protected from light at 4-8° C.

Lipid Stock

| Component | Amount |
|---|---|
| d-α-Tocopherol Acetate (d-Vitamin E acetate; | 14 mg |
| SIGMA-ALDRICH Cat. No. T-1157) | |
| PROFLO Cottonseed Oil (TRADERS PROTEIN, | 100 mg |
| SOUTHERN COTTON OIL COMPANY, Memphis, | |
| Tennessee 38108, U.S.A.) | |
| Tween 80 (SIGMA-ALDRICH Cat. No. P-1754) | 525 mg |

Lipid Stock was prepared by adding each component to a 50 mL polypropylene tube, further adding absolute ethanol to a 10 ml volume, mixing by vortexing, and incubating the mixture in a 37° C. water bath for 6-7 hours with occasional vortexing. After 6-7 hours, the mixture (although hazy) was filtered through a Supor Acrodisc PF syringe filter (0.8 μm/0.2 μm; GELMAN 4187) and stored at less than 0° C.

For eRDF-ACF Medium containing 1× lipid concentration, 50 μL Lipid Stock was added per Liter of Medium (for final concentrations per Liter of: 0.07 mg d-Vitamin E; 0.5 mg Cottonseed Oil; and 2.625 mg Tween 80). For eRDF-ACF Medium containing 10× lipid concentration, 500 μL Lipid Stock was added per 1 Liter of Medium (for final concentrations per Liter of: 0.7 mg d-Vitamin E; 5.0 mg Cottonseed Oil; and 26.25 mg Tween 80).

Medical Antifoam C Emulsion (10% w/w in picopure distilled H₂O and autoclaved 30 minute; DOW CORNING Cat. No. 1456211).

Pluronic F-68 (10%) (100×) Autoclaved for 30 minutes (LIFE TECHNOLOGIES Cat No. 24040-032).

### eRDF-ACF Medium

| Component | Amount | | Conc. |
|---|---|---|---|
| Wheat Gluten Peptides | 7 | g | 6.6 g/L |
| ERDF Stock w/o HEPES Buffer | 1000 | mL | 0.95X |
| Glycerol | 1 | g | 0.095% |
| 2-mercaptoethanol Stock (1000X) | 1 | mL | 0.95X |
| L-Glutamine Stock (200 mM) | 10 | mL | 1.9 mM |
| IES Stock (100X) | 10 | mL | 0.95X |
| Ascorbic Acid Stock (5 mM) | 1 | mL | 4.7 μM |
| Ferric Citrate Stock (200 mM) | 10 | mL | 1.9 mM |
| Lipid Stock | 500 | μL | 4.7 × 10⁻⁴X |
| Medical Antifoam C Emulsion | 1 | mL | 0.095% |
| Pluronic F-68 (10%) (100X) | 20 | mL | 1.9X |
| Approximate final volume | 1052 | mL | |

eRDF-ACF Medium was prepared as follows: a 7 g measure of powdered Wheat Gluten Peptides was placed in a sterile 1-L polystyrene storage bottle. A 1000 mL aliquot of liquid eRDF Stock w/o HEPES Buffer was pre-warmed to 37° C., added to the storage bottle, and mixed with a magnetic stir bar. Glycerol (1 g) was then added and dissolved. Next, all stock solutions, except Medical Antifoam C Emulsion and Pluronic F-68 Stock, were added, the combination was well mixed, and then filtered using a Millipore GP Express 0.22 μm vacuum filter unit. Finally, the Medical Antifoam C Emulsion and Pluronic F-68 Stock were added aseptically.

### EXAMPLE 2

### ELISA Quantification

ELISA plates were coated with goat anti-human Ig unlabelled antibody (SOUTHERN BIOTECHNOLOGY ASSOCIATES, Cat. No. 2010-01) and incubated at 4° C. overnight. After overnight incubation, the plates were emptied and washed with a BBS solution (i.e., 6.2 g/L Boric Acid (SIGMA-ALDRICH Cat. No. B-7660), 9.5 g/L Borax (SIGMA-ALDRICH Cat. No. B-9876), 4.4 g/L NaCl, pH adjusted to 8.2 to 8.4). Following the washes, 200-300 μL of BBS solution containing 1% albumin (BBS-BSA) was added to each well. Plates were incubated at room temperature for at least one hour or stored in a refrigerator with plate sealers. The plates were then emptied and washed with BBS solution. To the emptied plates, 100-200 μL of hybridoma supernatants, standards, their dilutions, and negative controls diluted in BBS-BSA were added per well. The plates were then incubated for three to four hours. After incubation, each plate was washed with BBS solution. The detecting antibody, goat anti-human alkaline phosphate (SOUTHERN BIOTECHNOLOGY ASSOCIATES, Cat. No. 2010-04), was added at 100 μL /well at a 1:500 dilution in BBS-BSA solution. The plates were incubated at room temperature for three to four hours and then washed with BBS solution. Thereafter, 100 μL/well of freshly prepared substrate was added to the wells. The substrate was prepared by dissolving SIGMA 104® Phosphatase Substrate (p-nitrophenyl phosphate; SIGMA-ALDRICH Cat. No. 104-40) to a final concentration of 1 mg/mL. The plates were incubated for one hour. The plates were then read on a microplate reader at a wavelength of 405 nanometers.

### EXAMPLE 3

### Development of Anti-CTLA4-Antibody-Producing Hybridomas for Culture in Serum-Free Medium

Hybridoma 4.1.1 was developed as described in published international patent application WO 00/37504. Hybridoma 4.1.1.1 is a sub-clone of hybridoma 4.1.1. Hybridoma 4.1.1.1 was cultured in a serum- and CS-containing medium. The CS in the medium was then replaced with murine IL6. Hybridoma 4.1.1.1 produced about 15 mg/L anti-CTLA4 antibody in batch culture, as assayed by enzyme-linked immunosorbent assay (ELISA).

Hybridoma 4.1.1.1 was weaned off serum and murine IL6 using traditional adaptation methods. This serum, CS, and IL-6 independent culture was renamed hybridoma 4C2. In a serum-free DMEM/F12-based medium (containing approximately 2415 mg/L sodium dibasic phosphate), hybridoma 4C2 also produced about 15 mg/L anti-CTLA4 antibody in batch culture when anti-CTLA4 antibody level was measured by ELISA.

Upon repeated runs of cloning by conventional limited serial dilution of hybridoma 4C2 cultures, a hybridoma cell line designated 0.5-10B was obtained for culture in a serum-free, DMEM/F12-based medium (containing approximately 1208 mg/L sodium dibasic phosphate). The format of the limited serial dilution protocol followed is as described in Example 4 for the development of hybridoma 0.5-G1. In the serum-free, DMEM/F12-based medium (containing approximately 1208 mg/L sodium dibasic phosphate), hybridoma 0.5-10B produced about 19 mg/mL anti-CTLA4 antibody in batch culture when anti-CTLA4 antibody level was measured by ELISA.

### EXAMPLE 4

### Development of Hybridoma 0.5-G1

Hybridoma 0.5-10B was then subcloned in a serum-free, eRDF-based medium (containing 0.95× eRDF Stock w/HEPES Buffer; LIFE TECHNOLOGIES Cat. No. 00-0019DK) by conventional limited serial dilution to obtain hybridoma 0.5-G1 as follows. Hybridoma 0.5-10B cells that had been stored in liquid nitrogen were thawed in a 37° C. water bath. Thawed cells were cultured in the serum-free, DMEM/F12-based medium (containing approximately 1208 mg/L sodium dibasic phosphate) on a schedule in which cell cultures were split every two days over the course of one week (i.e., cultures were split three times). On day eight, cultured cells were transferred to serum-free, eRDF-based medium supplemented with 1% L-Glutamine, 1×ITS-S, and 1% Pluronic F68 Stock. On day nine, a cell suspension (7.50×10⁵ cells/ml) was serially diluted using serum-free, eRDF-based medium (without supplements) into dilutions of 1:7.5, 1:37.5, 1:375, 1:3,750, 1:30,000, 1:75,000, 1:150,000, and 1:750,000; half of the medium used for dilutions to generate the four most dilute suspensions of this series was conditioned medium.

Cells from each of the two most dilute suspensions of this series were used to inoculate wells of 96-well plates to generate plates of 0.5 cell per well, and of 0.1 cell per well, where each well contained a suspension volume of 100 μL. These plates were placed in a super-humidified chamber and cultured at 5% CO₂ and 37° C. After two days, 100 μL fresh serum-free, eRDF-based medium was added to each of the wells on these plates in order to replenish nutrients.

Well suspensions generating high levels of human antibody were determined by ELISA as described in Example 2. Hybridoma cultures from wells producing high levels of human antibody were scaled up once a well reached confluence. Hybridomas from nine wells of plates of 0.5 cell per well and from three wells of plates of 0.1 cell per well had the highest levels of production of human antibody (i.e., anti-CTLA4 MAb) as measured by ELISA. To complete what was considered to represent one passage for these cells (i.e., growth in 96-well plates), the contents of each of these twelve wells were aseptically removed from the 96-well ELISA plates through gentle pipetting and transferred to a corresponding well on a 12-well incubation plate.

To help stimulate growth, 1 ml serum-free, eRDF-based medium additionally containing 0.95× Hybridoma Fusion and Cloning Supplement (BOEHRINGER MANNHEIM Cat. No. 1-363-735) was added to each of the 12 wells. After several days of culture, three wells were confluent. Gentle pipetting was used to transfer cells from these wells to T25 flasks containing 7.5 ml serum-free, eRDF-based medium without CS. Culture of the cell lines in 12-well plates was considered to represent passage two for these cell lines.

Cell line 0.5-G1 was chosen for continued cloning in additional passages in flasks. An aliquot of cell line 0.5-G1 was cultured in serum-free, eRDF-based medium in a 250 ml shake flask in an incubator set at 130 rpm and 37° C. with 5% CO₂. After three days in culture, a 1 ml sample was taken from the flask for ELISA. One day later, a second 1 ml sample was taken from the flask for another ELISA. Culture of the original flask was maintained in order to obtain ELISA samples up to two days later.

ELISA performed according to standard protocols on such samples, as described in Example 2, determined that cell line 0.5-G1 initially produced about 73 mg human anti-CTLA4 antibody per liter at a density of 1.20×10⁶ cells/ml and at a cell viability of 57.1%. Because the antibody titer represented a significant increase over previous cell lines, cell line 0.5-G1 was cryopreserved in liquid nitrogen.

Hybridoma 0.5-G1 was later admixed with, and cultured in, eRDF-ACF Medium, which is a serum-free and animal-components-free medium. In eRDF-ACF Medium, 0.5-G1 hybridoma produced levels of anti-CTLA4 antibody in batch culture of greater than 200 mg/L (and up to about 260 mg/L) when anti-CTLA4 antibody level was measured by ELISA.

### EXAMPLE 5

### Development of Hybridoma 5d3

Upon another run of cloning by conventional limited serial dilution of hybridoma 0.5-G1 cultures and growth in eRDF-ACF Medium, a hybridoma cell line designated 5D3 was obtained. The format of the limited dilution protocol followed was as described in Example 4. In eRDF-ACF Medium, hybridoma 5D3 produced higher levels of anti-CTLA4 antibody (e.g., greater than 200 mg/L, generally greater than 300 mg/L, and up to about 390 mg/L) in batch culture when anti-CTLA4 antibody level was measured by ELISA. Hybridoma 5D3 was deposited with the American Type Culture Collection (ATCC), 10801 University Boulevard, Manassas, Va. 20110-2209ATCC on Jul. 9, 2002, and was accorded a deposit number of PTA-4537.

1. A hybridoma, wherein said hybridoma produces at least 200 mg/L of a human sequence antibody when cultured in batch culture.

2. The hybridoma of claim 1, wherein said hybridoma produces at least 300 mg/L of said antibody.

3. The hybridoma of claim 1, wherein said hybridoma produces at least 390 mg/L of said antibody.

4. The hybridoma of claim 1 wherein the antibody is an anti-CTLA4 antibody.

5. The hybridoma of claim 4 having the identifying characteristics of the hybridoma deposited at ATCC deposit number PTA-4537.

6 The hybridoma of claim 1 that is derived from the hybridoma deposited at ATCC deposit number PTA-4537.

7. The hybridoma of claim 1, wherein the hybridoma is a murine hybridoma.

8. An antibody produced by the hybridoma of claim 1.

9. A cell culture comprising a hybridoma producing an antibody having a human amino acid sequence in animal components-free medium that produces at least 200 mg/L of said human sequence antibody.

10. A cell culture of claim 9 that produces at least 300 mg/L of said antibody.

11. A method of producing antibody comprising culturing the cell culture of claim 10 under conditions effective for production of said antibody and isolating said antibody from said culture.

12. An antibody produced by the method of claim 11.

13. A medium free of animal-derived components, the medium comprising 2 to 10 g/L Wheat Gluten Peptides, 0.85 to 0.99× eRDF Stock without HEPES Buffer, 0 to 0.5% Glycerol, 0 to 2× 2-mercaptoethanol Stock, 0.5 to 8 mM L-Glutamine, 0.5 to 5×IES Stock, 2 to 8 μM Ascorbic Acid, 1 to 5 mM Ferric Citrate, 2 to 10×10⁻⁴× Lipid Stock, 0 to 0.5% Medical Antifoam C Emulsion, and 0 to 4× Pluronic F-68 (10% Stock=100×).

14. A pharmaceutical composition for the treatment of cancer in a mammal comprising an amount of an anti-CTLA4 antibody of claim 8 or 12 that is effective in treating said cancer and a pharmaceutically acceptable carrier.

15. A method for the treatment of cancer in a mammal comprising administering to said mammal an amount of an antibody of an anti-CTLA4 antibody of claim 8 or 12 that is effective in treating said cancer.
